(19) United States
(12) Patent Application Publication
(10) Pub. No.: US 2019/0183347 A1
(43) Pub. Date: Jun. 20, 2019

(54) PHOTOACOUSTIC APPARATUS AND OBJECT INFORMATION ACQUIRING METHOD

(71) Applicant: CANON KABUSHIKI KAISHA, Tokyo (JP)

(72) Inventor: Yoshihiro Hirata, Fuchu-shi (JP)

(21) Appl. No.: 16/220,473

(22) Filed: Dec. 14, 2018

(30) Foreign Application Priority Data
Dec. 20, 2017 (JP) 2017-244271

(51) Int. Cl. A61B 5/00; A61B 8/00; A61B 8/08

(52) U.S. Cl. CPC A61B 5/0095; A61B 5/0059; A61B 8/483; A61B 8/429; A61B 8/4281

(57) ABSTRACT

A photoacoustic apparatus is the photoacoustic apparatus that receives an acoustic wave, which is generated from an object irradiated with light, at a plurality of relative positions with respect to the object using a conversion element, and acquires a photoacoustic image representing information on the object, the photoacoustic apparatus including: an imaging unit configured to acquire an optical image of the object; and a positional alignment unit configured to align positions of the photoacoustic image and the optical image based on positional relationship information, which is information representing a positional relationship between a first coordinate system in the optical image and a second coordinate system in the photoacoustic image.

### BACKGROUND OF THE INVENTION

### Field of the Invention

The present invention relates to a photoacoustic apparatus.

### Description of the Related Art

As a technique to image functional information, such as structural information and physiological information, inside an object, photoacoustic imaging is known.

When light, such as laser light, is irradiated to a living body (object), an acoustic wave (typically an ultrasonic wave) is generated in response to an absorption of the light by a biological tissue inside the object. This phenomenon is called a “photoacoustic effect”, and the acoustic wave generated due to the photoacoustic effect is called a “photoacoustic wave”. The tissues constituting the object have different absorption rates of optical energy, hence the generated photoacoustic waves also have different sound pressures. With PAI (Photo Acoustic Imaging), a generated photoacoustic wave is received by a probe, and the received signal is mathematically analyzed, whereby characteristic information inside the object can be acquired.

For example, International Publication No. 2010/030817 discloses an apparatus, in which a plurality of conversion elements which converts an acoustic wave generated inside an object into electric signals, is disposed on a hemispherical support member. In this apparatus, the object is held by a thin cup type holding member, and an acoustic medium to match the acoustic impedance is disposed between the holding member and the conversion elements. Light is irradiated to the object from the lower part of the support member via the holding member and the acoustic medium, and the acoustic wave generated in the object reaches the conversion elements via the holding member and the acoustic medium.

### SUMMARY OF THE INVENTION

In a case where a user compares a photoacoustic image with an object, in some cases the positional relationship between the photoacoustic image and the actual object may not be clearly recognized. For example, the surface of a femoral region does not have distinctive characteristics, unlike the palms, hence it may be difficult to recognize the correspondence between the observing region and the actual part of the femoral region by an angiogram alone.

With the foregoing problem of the prior art in view, it is an object of the present invention to specify a captured position of a photoacoustic image on the object.

To solve this problem, a photoacoustic apparatus according to the present invention is the photoacoustic apparatus that receives an acoustic wave, which is generated from an object irradiated with light, at a plurality of relative positions with respect to the object using conversion elements, and acquires a photoacoustic image representing information on the object, the photoacoustic apparatus including: an imaging unit configured to acquire an optical image of the object; and a positional alignment unit configured to align positions of the photoacoustic image and the optical image based on positional relationship information, which is information representing a positional relationship between a first coordinate system in the optical image and a second coordinate system in the photoacoustic image.

An object information acquiring method according to the present invention is the object information acquiring method performed by a photoacoustic apparatus that includes conversion elements configured to receive an acoustic wave, which is generated from an object irradiated with light, and convert the acoustic wave into a photoacoustic signal, the object information acquiring method including: a scanning step of moving the conversion elements relatively to the object; a first acquiring step of acquiring a photoacoustic image, which represents information on the object, based on the photoacoustic signal; an imaging step of imaging the object by an imaging unit and acquiring an optical image; a second acquiring step of acquiring positional relationship information, which is information representing a positional relationship between a first coordinate system in the optical image and a second coordinate system in the photoacoustic image; and a positional alignment step of aligning positions of the photoacoustic image and the optical image based on the positional relationship information.

According to the present invention, a captured position of a photoacoustic image can be specified on the object.

Further features of the present invention will become apparent from the following description of exemplary embodiments with reference to the attached drawings.

### BRIEF DESCRIPTION OF THE DRAWINGS

FIG. 1 is a schematic diagram depicting a photoacoustic apparatus according to Embodiment 1;

FIG. 2 is a diagram depicting optical markers captured in an optical image;

FIG. 3 is a flow chart depicting processing performed by the photoacoustic apparatus according to Embodiment 1;

FIGS. 4A to 4C are examples of an optical image and a photoacoustic image;

FIG. 5 is a schematic diagram depicting a photoacoustic apparatus according to Embodiment 2;

FIG. 6 is a flow chart depicting processing performed by the photoacoustic apparatus according to Embodiment 2;

FIG. 7 is a diagram depicting a method of combining a plurality of optical images;

FIG. 8 is a schematic diagram depicting a photoacoustic apparatus according to Embodiment 3; and

FIG. 9 is a flow chart depicting processing performed by the photoacoustic apparatus according to Embodiment 3.

### DESCRIPTION OF THE EMBODIMENTS

Embodiments of the present invention will be described with reference to the drawings. Dimensions, materials, shapes and relative positions of the components described below should be appropriately changed depending on the configurations and various conditions of the apparatus to which the invention is applied. Therefore, the following description is not intended to limit the scope of the invention.

The present invention relates to a technique to detect an acoustic wave propagating from an object, and generate and acquire the characteristic information inside the object. This means that the present invention is regarded as a photoacoustic apparatus or a control method thereof. The present invention is also regarded as a program which causes the apparatus, equipped with such hardware resources as a CPU and a memory, to execute this method, or a computer readable non-transitory storage medium storing this program.

The photoacoustic apparatus according to the embodiments is an apparatus utilizing a photoacoustic effect, that is, an acoustic wave generated inside an object by irradiating light (electromagnetic wave) to the object is received, and characteristic information of the object is acquired as the image data. In this case, the characteristic information refers to information on the characteristic values corresponding to a plurality of positions inside the object respectively, and these characteristic values are generated using the received signals which are acquired by receiving a photoacoustic wave.

The characteristic information acquired by the photoacoustic measurement refers to the values reflecting the absorption rate of the optical energy. For example, the characteristic information includes a generation source of an acoustic wave which was generated by the light irradiation, an initial sound pressure inside the object, an optical energy absorption density and an absorption coefficient derived from the initial sound pressure, and a concentration of a substance constituting a tissue.

Based on photoacoustic waves that are generated by lights having a plurality of different wavelengths, spectral information, such as the concentration of a substance constituting the object, can be acquired. The spectral information may be an oxygen saturation, a value generated by weighting the oxygen saturation by intensity (e.g. absorption coefficient), a total hemoglobin concentration, an oxyhemoglobin concentration or a deoxyhemoglobin concentration. The spectral information may also be a glucose concentration, a collagen concentration, a melanin concentration, or a volume fraction of fat or water.

In the following embodiments described below, it is assumed that a photoacoustic imaging apparatus is used and configured to acquire data on distribution and profiles of blood vessels inside the object and data on the oxygen saturation distribution in the blood vessels, by irradiating light, having a wavelength which is determined based on the assumption that the absorber is hemoglobin, to the object.

Based on the characteristic information at each position inside the object, a two-dimensional or three-dimensional characteristic information distribution is acquired. The distribution data can be generated as image data. The characteristic information may be determined, not as numeric data, but as distribution information at each position inside the object. In other words, such distribution information as the initial sound pressure distribution, the energy absorption density distribution, the absorption coefficient distribution and the oxygen saturation distribution may be acquired.

The “acoustic wave” in the present description is typically an ultrasonic wave, including an elastic wave called a “sound wave” or a “photoacoustic wave”. An electric signal, converted from an acoustic wave by a probe or the like, is called an “acoustic signal”. Such phrases as “ultrasonic wave” or “acoustic wave” in this description, however, are not intended to limit the wavelengths of these elastic waves. An acoustic wave generated due to the photoacoustic effect is called a “photoacoustic wave” or a “optical ultrasonic wave”. An electric signal, which originates from a photoacoustic wave, is called a “photoacoustic signal”. In this description, the photoacoustic signal includes both an analog signal and a digital signal. The distribution data is also called “photoacoustic image data” or “reconstructed image data”.

The photoacoustic apparatus according to the embodiments is an apparatus that irradiates a pulsed light to an object and receives a photoacoustic wave generated inside the object, so as to generate information related to the optical characteristic inside the object.

In the case of a skin flap sampling operation, acquiring a photoacoustic image (angiogram) is considered by performing the photoacoustic measurement of the skin flap sampling region, and detecting the positions and running state based on this angiogram, so as to determine the position and the range of the skin flap to be sampled. In this case, the operator must recognize the correspondence between the angiogram and a section of the object. However, for the skin flap sampling, such a region as a femoral region, which is relatively wide and has little characteristic in the body surface form, is normally used. Therefore, it tends to be difficult to specify the positional relationship between the angiogram and the actual object.

Therefore, in the following examples, the position in the object where the photoacoustic image corresponds to is clearly presented to the operator, so as to easily recognize the correspondence between the photoacoustic image and the object. Thereby the operator can recognize the actual position of the blood vessel (e.g. perforator branch) in the object based on the angiogram, and can sample the skin flap easily.

### Embodiment 1

### System Configuration

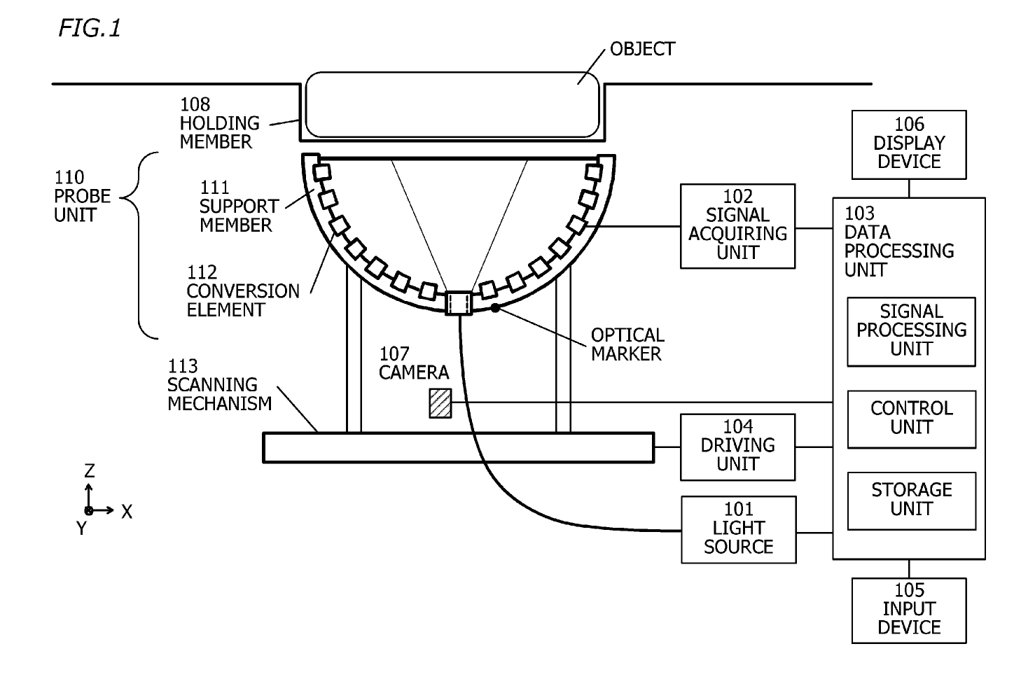

FIG. 1 is a schematic diagram depicting a configuration of a photoacoustic apparatus according to Embodiment 1. The photoacoustic apparatus according to Embodiment 1 is constituted of a light source 101, a signal acquiring unit 102, a data processing unit 103, a driving unit 104, an input device 105, a display device 106, a camera 107 and a probe unit 110. The probe unit 110 is constituted of a support member 111, a conversion element 112, and a scanning mechanism 113.

The probe unit 110 is a unit configured to irradiate light to an object and receive a generated acoustic wave from the object. The probe unit 110 is configured by disposing a plurality of conversion elements 112 in a spiral on an inner surface of a hemispheric support member 111. Further, a member to emit light irradiated from the later mentioned light source 101 is disposed on the base of the support member 111.

The support member 111 is an approximately hemispheric-shaped container that supports the plurality of conversion elements 112. In Embodiment 1, the plurality of conversion elements 112 are disposed on the inner surface of the hemisphere, and a member to emit light is installed at the base (pole) of the hemisphere. Acoustic matching material (e.g. water) may be stored inside the hemisphere. To support these members, it is preferable that the support member 111 is constituted of a metal material or the like which has high mechanical strength.

The conversion element 112 is a unit that receives an acoustic wave coming from inside the object and converts the received acoustic wave into an electric signal. The conversion element is also called an acoustic wave detector, an acoustic wave receiver or a transducer.

An acoustic wave generated from a living body is an ultrasonic wave in the 100 KHz to 100 MHz range, hence an element that can receive this frequency band is used for the acoustic wave detector. In concrete terms, a transducer utilizing a piezoelectric phenomenon, a transducer utilizing the resonance of light, a transducer utilizing the change in capacitance or the like can be used.

It is preferable that the conversion element 112 has high sensitivity and can handle a wide frequency band. In concrete terms, a piezoelectric element using lead zirconate titanate (PZT) or an element using a high polymer piezoelectric material, such as polyvinylidene fluoride (PVDF), a capacitive micro-machine ultrasonic transducer (CMUT), a Fabry-Perot interferometer or the like may be used. The conversion element 112, however, is not limited to the above, but may be any element as long as the functions of the probe can be performed.

The plurality of conversion elements 112 are arranged in an array on the hemispheric surface, so that the receiving directions of the elements are directed to the center of the curvature of the hemisphere. By disposing the plurality of conversion elements 112 like this, high resolution can be implemented at the center of curvature of the hemisphere.

The probe unit 110 can be moved in three-dimensional directions by the scanning mechanism 113. Thereby the light irradiating position and the acoustic wave receiving position can be relatively moved with respect to the object. The scanning mechanism 113 may have a guide mechanism, a driving mechanism and a scanning position sensor in the three directions of X, Y and Z axes respectively. The position of the probe unit 110 can be expressed by the position of the scanning mechanism 113.

The light source 101 is a unit configured to generate a pulsed light which is irradiated to an object. The light source is preferably a laser light source in order to generate high power, but a light-emitting diode or a flash lamp may be used instead of laser. In the case of using a laser as the light source, various lasers, such as a solid-state laser, a gas laser, a dye laser, and a semiconductor laser can be used. For example, an Nd: YAG laser, an alexandrite laser, a Tisa laser, an OPO laser or the like may be used.

The wavelength of the pulsed light is preferably a specific wavelength that is absorbed by a specific component, out of the components constituting the object, and is also a wavelength by which the light can propagate into the object. In concrete terms, a wavelength from 600 nm to 1100 nm is preferable. The light in this range can reach a relatively deep region of the living body, hence information in the deep region of the object can be acquired.

To effectively generate the photoacoustic wave, the light must be irradiated for a sufficiently short time, in accordance with the thermal characteristic of the object. In a case where the object is a living body, as in the case of Embodiment 1, the pulse width of the pulsed light that is generated from the light source is preferably 10 to 100 nanoseconds.

The timing, waveform, intensity and the like of the light irradiation are controlled by the later mentioned data processing unit 103.

The light emitted from the light source is guided to the object, while being processed to a predetermined light distribution profile by such optical components as a lens and mirror and is irradiated. The light may be propagated by an optical wave guide, such as optical fiber.

The optical system may include such optical components as a lens, a mirror, a prism, an optical fiber, a diffusion plate, a shutter and a filter. Any optical component may be used for the optical system, as long as the light emitted from the light source can be irradiated in a desired profile to the object. In terms of the safety of the living body and expanding the diagnostic region, it is preferable to expand the light to a certain area, rather than condensing the light by a lens.

The holding member 108 is a member that holds an object. In Embodiment 1, the object is inserted in the positive direction on the Z axis in FIG. 1 and is held in a state of being in contact with the holding member 108. It is preferable that the holding member 108 is constituted of a material having strength to support the object and a characteristic to transmit light and acoustic waves, such as polyethylene terephthalate. If necessary, an acoustic matching material may be stored inside the holding member 108.

The signal acquiring unit 102 is a unit that amplifies an electric signal acquired by the conversion element 112 and converts the electric signal into a digital signal.

The signal acquiring unit 102 may be configured by an amplifier that amplifies a received signal, an A/D convertor that converts the received analog signal into a digital signal, a memory (e.g. FIFO) that stores the received signal, and an arithmetic circuit (e.g. FPGA chip). Further, the signal acquiring unit 102 may be configured by a plurality of processors and arithmetic circuits.

The data processing unit 103 is a unit (control unit) that controls each composing element of the photoacoustic apparatus. For example, the data processing unit 103 controls the entire apparatus, such as control of the light irradiation to the object, the reception of an acoustic wave and photoacoustic signal, and the movement of the probe unit.

The data processing unit 103 is also a unit (signal processing unit) that acquires object information, such as the light absorption coefficient and oxygen saturation inside the object, based on the converted digital signal (photoacoustic signal). In concrete terms, the data processing unit 103 generates a three-dimensional initial sound pressure distribution inside the object from the collected electric signals.

The data processing unit 103 also generates a three-dimensional light intensity distribution inside the object, based on the information on the quantity of light irradiated to the object. The three-dimensional light intensity distribution can be acquired by solving the light diffusion equation using information on the two-dimensional light intensity distribution. Further, the data processing unit 103 can acquire the absorption coefficient distribution inside the object using the initial sound pressure distribution inside the object generated from the photoacoustic signals and the three-dimensional light intensity distribution. Furthermore, the data processing unit 103 can acquire the oxygen saturation distribution inside the object by computing the absorption coefficient distribution at a plurality of wavelengths.

The data processing unit 103 may have a function to perform desired processing, such as calculating the light intensity distribution, the information processing required for acquiring the optical coefficients of the background, and signal correction.

The data processing unit 103 may acquire instructions on changing the measurement parameters, starting and ending the measurement, selecting the image processing method, storing patient information and images, analyzing data and the like, via the later mentioned display device 106 and the input device 105.

The data processing unit 103 may be a computer constituted of a CPU, RAM, non-volatile memory and control port. In this case, the program stored in the non-volatile memory (storage unit) is executed by the CPU, whereby control is performed. The data processing unit 103 may be implemented by a general-purpose computer, or a dedicated workstation. The unit which plays an arithmetic function of the data processing unit 103 may be configured by a processor (e.g. CPU, GPU) and an arithmetic circuit (e.g. FPGA chip). These units may be a single processor or a single arithmetic circuit or may be constituted of a plurality of processors and arithmetic circuits.

The unit which plays a storage function of the data processing unit 103 may be a ROM, non-transitory storage medium (e.g. magnetic disk, flash memory), or a volatile medium (e.g. RAM). The storage medium storing the program is a non-transitory storage medium. Each of these units may be a single storage medium or may be constituted of a plurality of storage media. The unit which plays a control function of the data processing unit 103 may be configured by such an arithmetic element as a CPU.

The input device 105 is, for example, a pointing device (e.g. mouse, trackball, touch panel) and keyboard, but is not limited to these.

The display device 106 displays information acquired by the data processing unit 103 and the processed information thereof and is typically a display unit. The display device 106 may be a plurality of devices or may be a single device having a plurality of display sections so that parallel display is possible.

The camera 107 is a unit for observing an object and is typically a visible light camera which images the surface of the object. The camera 107 may be any camera as long as the surface of the object can be imaged. In Embodiment 1, a visible light camera, that can capture the entire measurement target region, is disposed at a position facing the object via the support member 111. An image captured by the camera 107 is hereafter called an “optical image”.

Overview of Photoacoustic Measurement

A method of measuring a living body (object) using the photoacoustic apparatus according to Embodiment 1 will be described next.

First a pulsed light emitted from the light source 101 is irradiated to an object via the optical system. When a part of the energy of the light propagating inside the object is absorbed by a light absorber (e.g. blood), an acoustic wave is generated from this light absorber by thermal expansion. If a cancer exists in a living body, light is uniquely absorbed by the newly generated blood vessels of the cancer, in the same manner as the case of blood in a normal region, and an acoustic wave is generated. The photoacoustic wave generated inside the living body is received by a plurality of conversion elements 112.

In Embodiment 1, the light is irradiated, and the acoustic wave is acquired by the scanning mechanism 113 changing the relative positional relationship between the support member 111 and the object. In other words, the photoacoustic signal can be acquired while light is irradiated a plurality of times to different positions on the object.

For example, while scanning the measurement target region of the object by a spiral or raster scan, irradiation of the pulsed light and reception of the photoacoustic wave are repeated at a predetermined cycle.

The signal received by the plurality of conversion elements 112 is converted by the signal acquiring unit 102 and is then sent to the data processing unit 103 as the photoacoustic signal. Parallel to this, the driving unit 104 sends the information on the irradiated position of the pulsed light to the data processing unit 103.

Based on the photoacoustic signal outputted from the signal acquiring unit 102 and the position information outputted from the driving unit 104, the data processing unit 103 reconstructs an image in order to acquire the characteristic distribution of the measurement target region. The characteristic distribution can be acquired as a set of voxel data in a case where three-dimensional information is acquired, or as a set of pixel data in a case where two-dimensional information is acquired. The acquired characteristic distribution becomes volume data representing the characteristic information (e.g. initial sound pressure distribution, absorption coefficient distribution) in the living body, and is converted into a two-dimensional image (photoacoustic image) and outputted via the display device 106.

In addition to these functions, the photoacoustic apparatus according to Embodiment 1 also has a function to output an optical image captured by the camera 107, together with the photoacoustic image. In concrete terms, the positions of the photoacoustic image and the optical image are aligned, and both these images are outputted in a superimposed state based on the result of this positional alignment. Thereby the user of the apparatus can recognize which part of the object the photoacoustic image corresponds to.

Superimposition of Photoacoustic Image and Optical Image

An overview of the processing unique to the photoacoustic apparatus according to Embodiment 1 will be described next.

The photoacoustic image is information on the optical characteristics inside the object, therefore, without processing, it is difficult to determine which part of the object this image corresponds. Hence the photoacoustic apparatus according to Embodiment 1 acquires information on the positional relationship (positional relationship information) between the coordinate system in the optical image (first coordinate system) and the coordinate system in the photoacoustic image (second coordinate system) in advance and aligns the positions of the photoacoustic image and the optical image using the acquired information.

Here processing to acquire the positional relationship between the coordinate system in the photoacoustic image and the coordinate system in the optical image will be described. The photoacoustic apparatus according to Embodiment 1 includes a plurality of conversion elements 112 in the probe unit 110, and can acquire a photoacoustic image in a predetermined range by one light irradiation. In other words, the position of the probe unit 110 (position of the scanning mechanism 113) and the range of the photoacoustic image are in association with each other. Therefore if the relationship between the optical image and the position of the probe unit 110 can be acquired, the coordinate system of the photoacoustic image and the coordinate system of the optical image can be corresponded.

In Embodiment 1, optical markers (position detecting markers) that can be read by the camera 107 are disposed on the rear surface of the support member 111, and the coordinate system of the photoacoustic image and the coordinate system of the optical image are corresponded using the positions of the optical markers detected in the optical image.

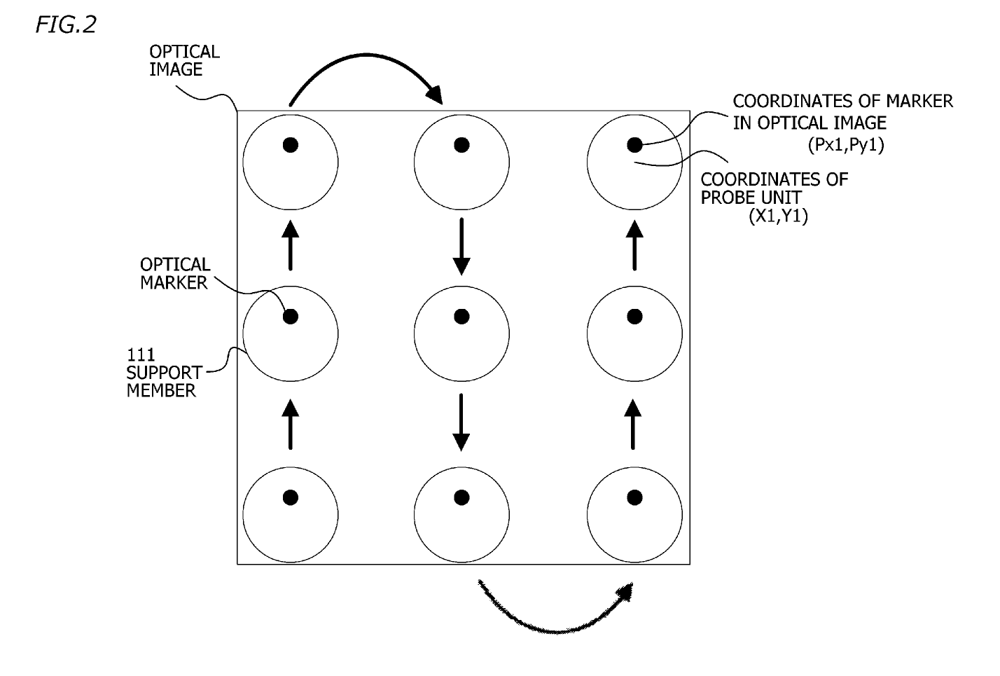

The processing flow will be described in concrete terms. FIG. 2 is an example of an optical image acquired by the camera 107. The optical image includes optical markers disposed in the support member 111. The optical markers are for acquiring the relative position of the probe unit with respect to the optical image and may be anything as long as it can be captured by the camera 107.

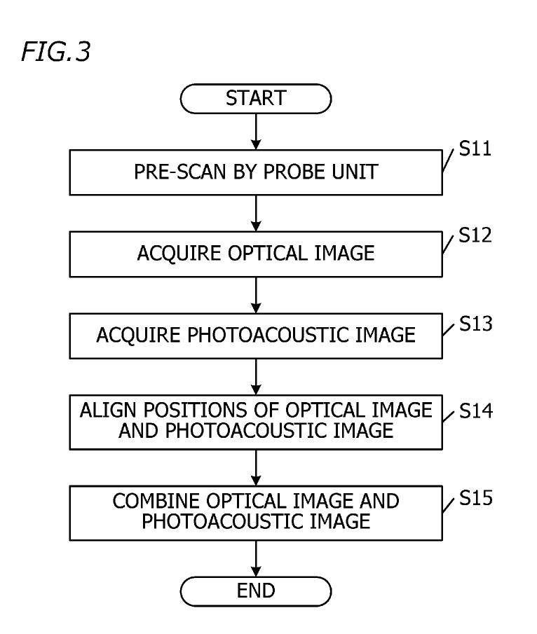

Prior to the photoacoustic measurement, the photoacoustic apparatus according to Embodiment 1 starts the processing in FIG. 3.

First, in step S11, the photoacoustic apparatus moves the probe unit 110 in a raster pattern using the scanning mechanism 113, as illustrated in FIG. 2, and detects the coordinates of the optical markers in the optical image. Then the position information (X, Y) of the probe unit 110 and the coordinates (Px, Py) of the optical markers in the optical image are stored in association with each other. In this example, the probe unit 110 is moved in a raster pattern, but the driving pattern is not limited to this. The coordinates to be stored may be coordinates of some of the representative points (representative positions). In this case, the coordinates between the representative points may be generated by linear interpolation or the like. The aberrations of the optical image may be complemented in the image processing in advance.

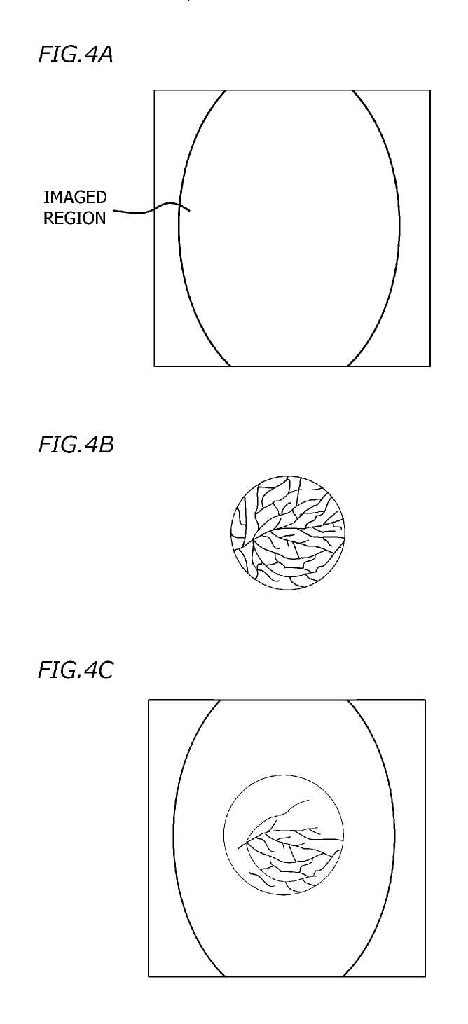

Then in step S12, the imaging region is imaged by the camera 107 so that the entire imaging range is included. The acquired optical image is sent to the data processing unit 103. FIG. 4A illustrates an image of the optical image.

Then in S13, the photoacoustic image is acquired by the abovementioned method. FIG. 4B illustrates an image of the photoacoustic image. The photoacoustic image and the corresponding irradiation position of the pulsed light are stored in association with each other.

Then in step S14, the data processing unit 103 (signal processing unit) aligns the positions of the photoacoustic image and the optical image based on the irradiation position of the pulsed light (coordinates of the probe unit 110), and the information on the coordinates of the optical markers in the optical image.

The coordinates of the photoacoustic image are associated with the irradiation coordinates of the pulsed light (coordinates of the probe unit 110). Further, the coordinates of the optical image and the coordinates of the probe unit 110 can be associated with each other based on the optical markers included in the optical image. In other words, the coordinate system in the optical image and the coordinate system in the photoacoustic image can be corresponded.

Finally in step S15, the images of which positions are aligned are combined to generate an image that is outputted to the display device 106. If necessary, the images may be resized or another image processing may be performed. FIG. 4C illustrates an image of the combined image. When the photoacoustic image and the optical image are combined, if the directions or positions of the object in the photoacoustic image and the optical image are different, rotating processing or moving processing may be performed at least on one of these images. For the processing, various known methods can be used.

As described above, according to Embodiment 1, the positional relationship of the photoacoustic image and the optical image is acquired, and then these images are combined. In other words, the user of the apparatus can accurately recognize the position on the object of which a photoacoustic image was captured.

### Embodiment 2

In Embodiment 1, the camera 107 is disposed in a position where the object can be imaged from the rear side of the support member 111. In Embodiment 2, on the other hand, the camera 107 is disposed on the probe (on the support member 111).

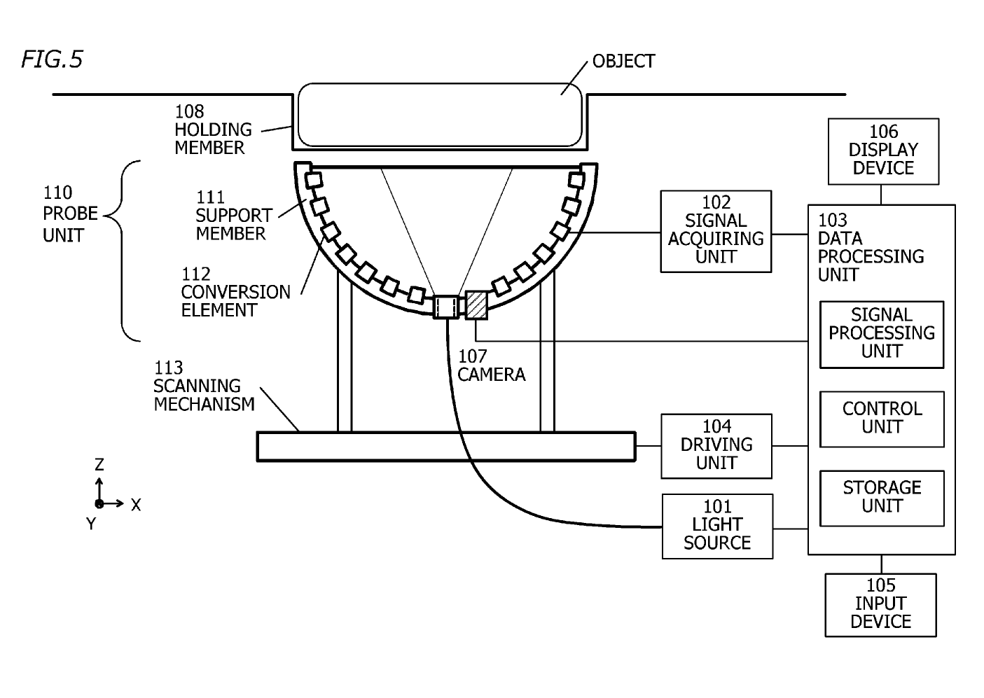

FIG. 5 is a schematic diagram depicting a configuration of a photoacoustic apparatus according to Embodiment 2.

In the photoacoustic apparatus according to Embodiment 1, the support member 111 and the camera 107 are separated, hence the probe unit 110 is retracted when the optical image is acquired. In the case of the photoacoustic apparatus according to Embodiment 2, on the other hand, the camera 107 is disposed on the support member 111, therefore the optical image can be acquired regardless the position of the probe unit 110.

If the camera 107 is disposed in this position, the distance between the imaging region and the camera becomes short, which makes the imaging area smaller and impossible to capture the entire imaging range in one optical image. Therefore, in Embodiment 2, a plurality of optical images are combined, whereby one optical image corresponding to the entire imaging range is generated.

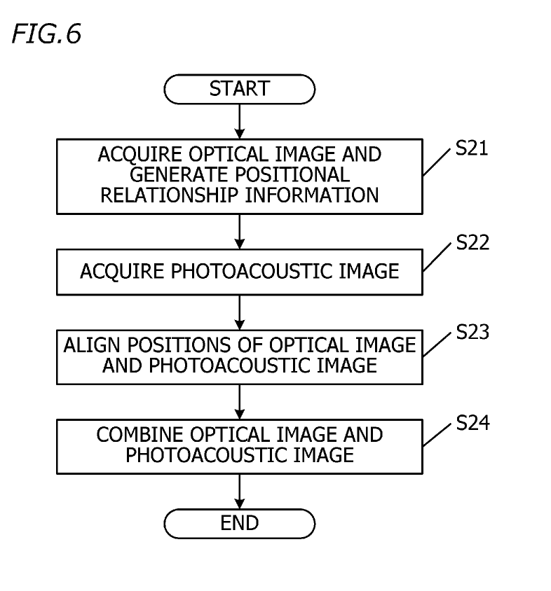

In Embodiment 2, the processing to acquire the positional relationship between the coordinate system in the photoacoustic image and the coordinate system in the optical image will be described. In Embodiment 2, the data processing unit 103 executes the processing indicated in FIG. 6 in a case where the photoacoustic measurement is performed.

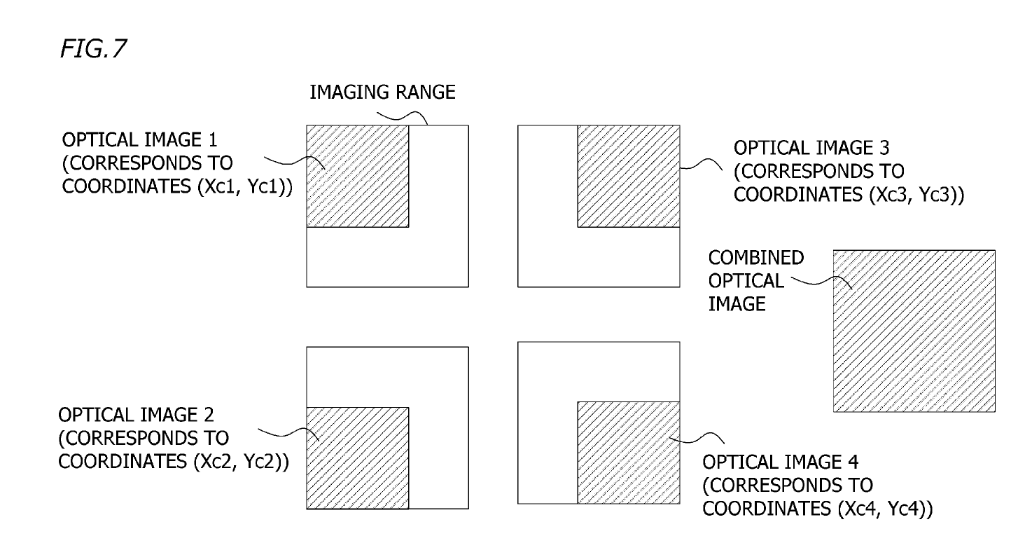

First, in step S21, the photoacoustic apparatus acquires a plurality of optical images while moving the probe unit 110 using the scanning mechanism 113 and combines these plurality of optical images so as to generate an optical image corresponding to the entire imaging range. As illustrated in FIG. 7, it is preferable to acquire an optical image at a plurality of positions with which the image ranges overlap. At this time, the coordinates (Xc, Yc) of the probe unit 110 are stored in association with the optical image. The optical images can be combined based on the coordinated positional information (Xc, Yc). Further, based on this information, the positional relationship information between the coordinate system of the photoacoustic image and the coordinate system of the optical image can be acquired.

Then in step S22, the photoacoustic image is acquired by the abovementioned method. At this time, the photoacoustic image and the corresponding irradiation position (Xp, Yp) of the pulsed light are stored in association with each other.

Then in step S23, the data processing unit 103 (signal processing unit) aligns the positions of the photoacoustic image and the optical image based on the irradiation position of the pulsed light (coordinates of the probe unit 110) and the positional relationship information acquired in step S21.

The coordinates of the photoacoustic image are associated with the irradiation position of the pulsed light (coordinates of the probe unit 110). Further, the coordinates of the optical image are also associated with the coordinates of the probe unit 110. Therefore, the coordinate system in the optical image and the coordinate system in the photoacoustic image can be associated.

Finally in step S24, the aligned images are combined to generate an image that is outputted to the display device 106. If necessary, the images may be resized or other image processing may be performed.

As described above, according to Embodiment 2, the camera 107 is disposed on the support member 111, hence the positional relationship information between the coordinate system in the photoacoustic image and the coordinate system in the optical image can be acquired, without reading the optical markers.

### Embodiment 3

In Embodiment 2, the object image is acquired at a timing before acquiring the photoacoustic image. In Embodiment 3, on the other hand, the optical image is acquired while performing the photoacoustic measurement.

In the case of Embodiment 2, the optical image is acquired separately before acquiring the photoacoustic image, hence both time for acquiring the optical image and time for acquiring the photoacoustic image are required. However, in the photoacoustic apparatus, the object is fixed so as to not move during the measurement, hence, if time for the measurement increases, the examinee experience additional discomfort.

To prevent this problem from occurring, in Embodiment 3, a trigger is generated by detecting a pulsed light irradiated to the object, and an optical image is acquired based on this trigger.

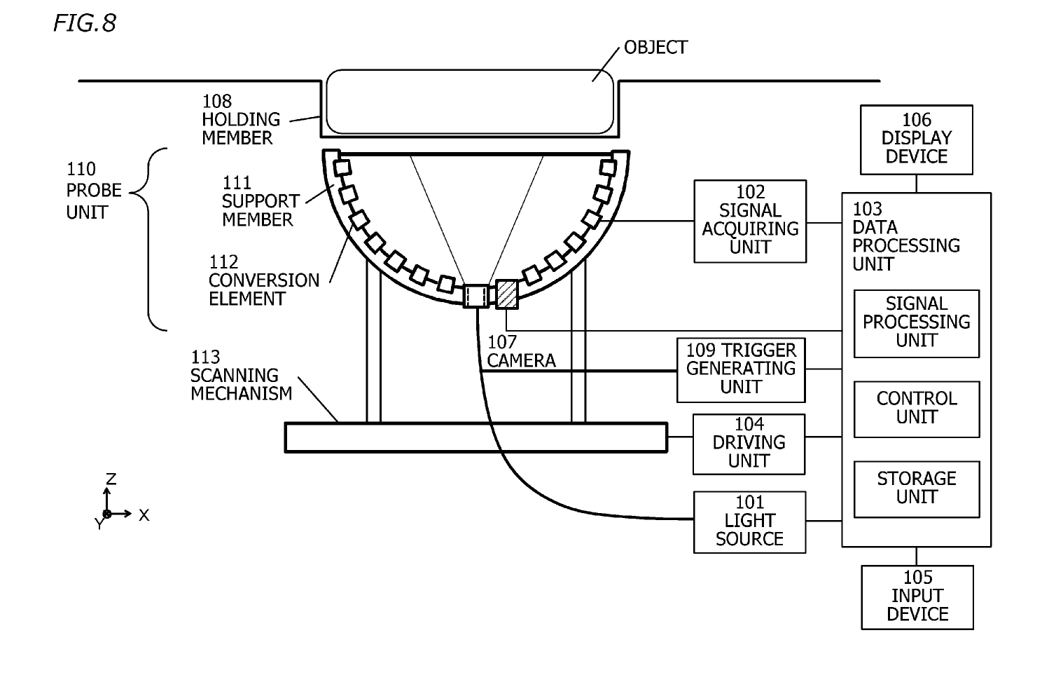

FIG. 8 is a schematic diagram depicting a configuration of a photoacoustic apparatus according to Embodiment 3.

The photoacoustic apparatus according to Embodiment 3 further includes a trigger generating unit 109 that detects light generated by the light source 101 and generates a trigger to acquire the optical image.

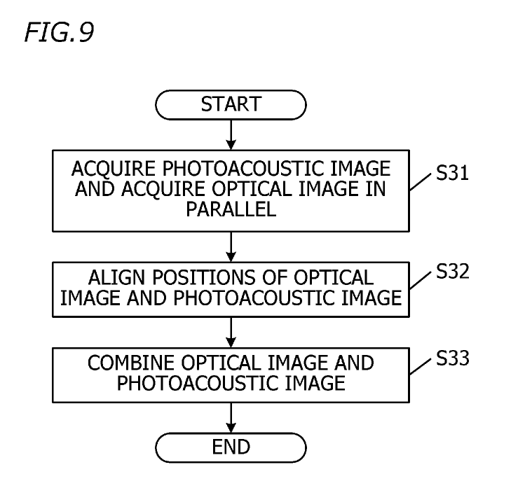

In Embodiment 3, the processing to acquire the positional relationship between the coordinate system in the photoacoustic image and the coordinate system in the optical image will be described. In Embodiment 3, the data processing unit 103 executes the processing in FIG. 9 in a case where the photoacoustic measurement is performed.

First, in step S31, the photoacoustic apparatus starts acquiring a photoacoustic image. In this step, the photoacoustic apparatus acquires photoacoustic signals by repeatedly irradiating the pulsed light and receiving the photoacoustic wave at a predetermined cycle, while moving the probe unit 100 using the scanning mechanism 113. At this time, the trigger generating unit 109 detects the pulsed light and generates a trigger signal, and the camera 107 acquires the optical image based on this trigger signal. It is preferable to acquire the optical image in a period from the end of the irradiation of the pulsed light to the start of the irradiation in the next cycle.

The emission cycle of the pulsed light is preferably set considering the time required for the scanning mechanism 113 to move to the next position.

In Embodiment 3, the positional information (Xpc, Ypc) of the pulse unit is stored in association with both the optical image and the photoacoustic image. Further, a plurality of optical images are combined based on this positional information, whereby an optical image corresponding to the entire imaging range is generated.

Then in step S32, the data processing unit 103 (signal processing unit) aligns the positions of the photoacoustic image and the optical image based on the positional information of the probe unit associated with the photoacoustic image and the optical image.

The coordinates of the photoacoustic image are associated with the coordinates of the probe unit 110. The coordinates of the optical image are also associated with the coordinates of the probe unit 110. Therefore, the coordinate system in the optical image and the coordinate system in the photoacoustic image can be associated.

Finally in step S33, the aligned images are combined to generate an image that is outputted to the display device. If necessary, the images may be resized or another image processing may be performed.

As described above, according to Embodiment 3, the photoacoustic image and the optical image are acquired in parallel, hence the measurement time can be decreased, and stress caused to the examinee can be reduced.

### Embodiment 4

In Embodiment 2, the coordinates of the optical image are associated with the coordinates of the probe unit 110, and the coordinates of the photoacoustic image are associated with the coordinates of the irradiation of the pulsed light, that is, the coordinates of the probe unit 110. In Embodiment 4, on the other hand, the coordinates of the optical image, the coordinates of the photoacoustic image, and the coordinates of the probe unit 110 are associated using time information.

A difference in the photoacoustic apparatus according to Embodiment 4 from that of Embodiment 2 is that the data processing unit 103 has a function to acquire the time information in the configuration illustrated in FIG. 5.

The data processing unit 103 acquires the time information on the timing when the camera 107 acquired the optical image and stores this information in the storage unit in association with the acquired optical image. In the same manner, the data processing unit 103 acquires the time information on the timing when the photoacoustic signal was acquired and stores the information in the storage unit in association with the acquired photoacoustic signal. Further, the data processing unit 103 acquires the coordinate information of the probe unit 110 at a predetermined timing and the time information on this timing, and stores both information in the storage unit in association with each other. Thereby the data processing unit 103 can associate the coordinates of the probe unit 110 at a timing when the optical image and each photoacoustic signal were acquired with the optical image and the photoacoustic signal, and as a result, positional alignment of the optical image and the photoacoustic image can be implemented.

The time information acquired by the data processing unit 103 may be a time information using GMT or may be unique time information used in the photoacoustic apparatus, or a time information based on certain processing executed by the photoacoustic apparatus.

Depending on the configuration of the apparatus, the deviation between the timing when the optical image is acquired and the timing when this time information is acquired may not match with the deviation between the timing when the photoacoustic signal was acquired and the timing when this time information was acquired. In such a case, the data processing unit 103 executes processing to compensate for this time difference, and then aligns the positions of the optical image and the photoacoustic image, whereby the accuracy of the positional alignment can be improved. The same is true for the deviation between the timing when the coordinate information of the probe unit 110 is acquired and the timing when this time information is acquired.

Concerning the processing procedure, the acquisition of the photoacoustic image and the acquisition of the optical image can be executed in parallel, just like Embodiment 3.

Modifications

The above description on each embodiment is an example to describe the present invention, and the present invention can be carried out by appropriately changing or combining the above embodiments within a scope that does not depart from the essence of the invention.

For example, the present invention may be carried out as a photoacoustic apparatus that includes at least a part of the abovementioned units. The present invention may also be carried out as an object information acquiring method that includes at least a part of the abovementioned processing. Further, the above processing and units may be freely combined within the scope of not generating technical inconsistencies.

In the description of the embodiments, the positional relationship information between the coordinate system in the optical image and the coordinate system in the photoacoustic image is generated in a case where the photoacoustic measurement is performed, but the positional relationship information may be provided from outside the apparatus. For example, the positional relationship information may be acquired via a network or may be acquired via a storage medium or the like. Further, the positional relationship information may have already been stored in the initial state.

In the examples described in the embodiments, the photoacoustic image and the optical image are displayed in a superimposed state, but these images need not be superimposed at all times if the positional relationship of these images is known. For example, the two images may be displayed side by side, so that the same region is displayed.

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2017-244271, filed on Dec. 20, 2017, which is hereby incorporated by reference herein in its entirety.

What is claimed is:

1. A photoacoustic apparatus that receives an acoustic wave, which is generated from an object irradiated with light, at a plurality of relative positions with respect to the object using conversion elements, and acquires a photoacoustic image representing information on the object, the photoacoustic apparatus comprising:
an imaging unit configured to acquire an optical image of the object; and
a positional alignment unit configured to align positions of the photoacoustic image and the optical image based on positional relationship information, which is information representing a positional relationship between a first coordinate system in the optical image and a second coordinate system in the photoacoustic image.

2. The photoacoustic apparatus according to claim 1, further comprising an output unit configured to output the photoacoustic image and the optical image after performing the positional alignment.

3. The photoacoustic apparatus according to claim 1, further comprising a combining unit configured to combine the photoacoustic image and the optical image based on the result of the positional alignment.

4. The photoacoustic apparatus according to claim 1, further comprising a generating unit configured to generate the positional relationship information based on positions of the conversion elements when the light is irradiated,
wherein the second coordinate system is associated with the positions of the conversion elements.

5. The photoacoustic apparatus according to claim 4, wherein
the conversion elements are disposed on a support member;
the imaging unit is disposed at a position facing the object via the support member;
a position detecting marker is disposed on the support member on the side facing the imaging unit; and
the generating unit generates the positional relationship information based on the position of the position detecting marker in the optical image.

6. The photoacoustic apparatus according to claim 4, wherein
the conversion elements are disposed on a support member; and
the imaging unit is disposed on the support member at a position facing the object.

7. The photoacoustic apparatus according to claim 6, wherein
the imaging unit acquires the optical image at a plurality of positions, and combines the plurality of optical images based on the positions of the conversion elements when the optical image was acquired.

8. An object information acquiring method performed by a photoacoustic apparatus that includes conversion elements configured to receive an acoustic wave, which is generated from an object irradiated with light, and convert the acoustic wave into a photoacoustic signal, the object information acquiring method comprising:
a scanning step of moving the conversion elements relatively to the object;
a first acquiring step of acquiring a photoacoustic image, which represents information on the object, based on the photoacoustic signal;
an imaging step of imaging the object by an imaging unit and acquiring an optical image;
a second acquiring step of acquiring positional relationship information, which is information representing a positional relationship between a first coordinate system in the optical image and a second coordinate system in the photoacoustic image; and
a positional alignment step of aligning positions of the photoacoustic image and the optical image based on the positional relationship information.

9. The object information acquiring method according to claim 8, further comprising an output step of outputting the photoacoustic image and the optical image after performing the positional alignment.

10. The object information acquiring method according to claim 8, further comprising a combining step of combining the photoacoustic image and the optical image based on the result of the positional alignment.

11. The object information acquiring method according to claim 8, further comprising a generating step of generating the positional relationship information based on positions of the conversion elements when the light is irradiated,
wherein the second coordinate system is associated with the positions of the conversion elements.

12. The object information acquiring method according to claim 11, wherein
the conversion elements are disposed on a support member;
the imaging unit is disposed at a position facing the object via the support member;
a position detecting marker is disposed on the support member on the side facing the imaging unit; and
in the generating step, the positional relationship information is generated based on the position of the position detecting marker in the optical image.

13. The object information acquiring method according to claim 11, wherein
the conversion elements are disposed on a support member; and
the imaging unit is disposed on the support member at a position facing the object.

14. The object information acquiring method according to claim 13,
wherein in the imaging step, the optical image is acquired at a plurality of positions, and the plurality of optical images are combined based on the positions of the conversion elements when the optical image was acquired.
